(19) United States
(12) Patent Application Publication
(10) Pub. No.: US 2019/0365355 A1
(43) Pub. Date: Dec. 5, 2019

(54) Sparsity-based ultrasound super-resolution imaging

(71) Applicant: TECHNION RESEARCH & DEVELOPMENT FOUNDATION LTD., Haifa (IL)

(72) Inventors: Yonina Eldar, Haifa (IL); Avinoam Bar-Zion, Haifa (IL); Oren Solomon, Ashdod (IL)

(21) Appl. No.: 16/478,480

(22) PCT Filed: Jan. 16, 2018
(86) PCT No.: PCT/IB2018/050254

§ 371 Date: Jul. 17, 2019

Related U.S. Application Data
(60) Provisional application No. 62/447,670, filed on Jan. 18, 2017.

(51) Int. Cl. A61B 8/00; A61B 8/08; A61B 8/06; G06T 3/40

(52) U.S. Cl. CPC A61B 8/463; A61B 8/481; A61B 8/0891; G06T 3/4053; A61B 8/06; A61B 8/5207; A61B 8/488

(57) ABSTRACT

An apparatus (20) for imaging includes an input interface (54) and a processor (50). The input interface receives a sequence of input images of a target. Each input image includes a grid of pixels representing reflections of a transmitted signal from reflectors or scatterers in the target. A resolution of the input images is degraded by a measurement process of capturing the input images in the sequence. The processor derives, from the sequence of input images, an aggregated image in which each pixel comprises a statistical moment calculated over corresponding pixels of the input images, and converts the aggregated image into a super-resolution image of the target, having a higher resolution than the input images, by applying to the aggregated image a recovery function, which outputs the super-resolution image as a solution to the recovery function, provided that the reflectors or scatterers are sparse or compressible in a predefined transform domain.

### CROSS-REFERENCE TO RELATED APPLICATIONS

This application claims the benefit of U.S. Provisional Patent Application 62/447,670, filed Jan. 18, 2017. This application is related to a U.S. patent application entitled “Sparsity-based super-resolution correlation microscopy,” Attorney Docket No. 1064-1028.1, filed on even date. The disclosures of these related applications are incorporated herein by reference.

### TECHNICAL FIELD

Embodiments described herein relate generally to imaging techniques, and particularly to methods and systems for ultrasound super-resolution imaging.

### BACKGROUND

Certain medical treatment and diagnostic procedures require high resolution imaging. For example, anticancer and anti-inflammatory treatments require the detection of possible changes caused to blood vessels at a microvascular level.

Contrast-Enhanced Ultrasound (CEUS) is an imaging technique in which Ultrasound Contrast Agents (UCAs) such as encapsulated microbubbles are injected into the blood stream. CEUS imaging is described, for example, by Hudson et al., in “Dynamic contrast enhanced ultrasound for therapy monitoring,” European Journal of Radiology, volume 84, number 9, 2015, pages 1650-1657.

Imaging spatial resolution is typically limited by the inherent Point-Spread Function (PSF) of the overall scanning system. Super-localization techniques that improve spatial resolution beyond PSF limitations were originally used in optical fluorescent microscopy, as described, for example, by Betzig et al. in “Imaging intracellular fluorescent proteins at nanometer resolution,” Science, volume 313, number 5793, 2006, pages 1642-1645. Applying super-localization to ultrasound imaging is described, for example, by Errico et al., in “Ultrafast ultrasound localization microscopy for deep super-resolution vascular imaging,” Nature, volume 527, number 7579, 2015, pages 499-502. CEUS super-localization typically requires long acquisition time, which is unacceptable in certain clinical situations, e.g., in detecting fast hemodynamic changes or in cases of rapid tissue and organ motion.

An approach for improving spatial resolution with short acquisition times was presented by Bar-Zion et al., in “Fast Vascular Ultrasound Imaging with Enhanced Spatial Resolution and Background Rejection,” IEEE Transactions on Medical Imaging, volume 7, 2016, pages 1-12. This approach was inspired by an optical fluorescence microscopy method denoted Super-Resolution Optical Fluctuation Imaging (SOFI), which is described, for example, by Dertinger et al., in “Fast, background-free, 3D super-resolution optical fluctuation imaging (SOFI),” Proceedings of the National Academy of Sciences of the United States of America, volume 106, number 52, 2009, pages 22287-22292.

### SUMMARY

An embodiment of the present invention that is described herein provides an apparatus for imaging including an input interface and a processor. The input interface is configured to receive a sequence of input images of a target. Each input image includes a grid of pixels representing reflections of a transmitted signal from reflectors or scatterers in the target. A resolution of the input images is degraded by a measurement process of capturing the input images in the sequence. The processor is configured to derive, from the sequence of input images, an aggregated image in which each pixel comprises a statistical moment calculated over corresponding pixels of the input images, and to convert the aggregated image into a super-resolution image of the target, having a higher resolution than the input images, by applying to the aggregated image a recovery function, which outputs the super-resolution image as a solution to the recovery function, provided that the reflectors or scatterers are sparse or compressible in the target in a predefined transform domain.

In some embodiments, the target includes a vasculature of an organ, and the reflectors or scatterers include one or more of (i) microbubbles administered into the vasculature and (ii) red blood cells flowing within the vasculature. In an embodiment, the processor is configured to convert the aggregated image into the super-resolution image so that reflectors or scatterers corresponding to overlapping echoes appear visually separated in the super-resolution image.

In an embodiment, the processor is configured to (i) derive from the sequence of input images multiple Doppler-band-specific image sequences, based on identifying multiple respective ranges of microbubble velocities, (ii) aggregate each of the Doppler-band-specific image sequences to produce a Doppler-band-specific aggregated image, (iii) convert each of the Doppler-band-specific aggregated images into a respective Doppler-band-specific super-resolution image, and (iv) reconstruct the super-resolution image of the target from the multiple Doppler-band-specific super-resolution images. In another embodiment, the processor is configured to transform the aggregated image from a spatial domain to a transform domain using a predefined transform, and to apply the recovery function to the aggregated image in the transform domain.

In some embodiments, the aggregated image and the super-resolution image are interrelated using a model that depends on a Point Spread Function (PSF) included in the measurement process. In an example embodiment, the processor is configured to estimate the PSF by identifying in the input images regions corresponding to non-overlapping echoes of the reflectors or scatterers, and estimating the PSF based on the identified regions. In another embodiment, the model includes an underdetermined linear model, the predefined recovery function includes a convex optimization problem based on the linear model, and the processor is configured to solve the convex optimization problem under a sparsity constraint.

In an embodiment, a matrix formulating the linear model has a Block Circulant with Circulant Blocks (BCCB) structure, and the processor is configured to solve the convex optimization problem by performing a sequence of iterations, and based on the BCCB structure, to calculate in each iteration a gradient value of a function derived from the linear model using FFT-based operations. The optimization problem may be formulated under a Total Variation (TV) constraint. In another embodiment, the optimization problem is formulated in a selected domain in which the solution is sparse and wherein the processor is configured to solve the optimization problem in the selected domain.

In some embodiments, the input interface is configured to receive multiple sequences of the input images over multiple respective scanning cycles, and the processor is configured to produce multiple respective super-resolution images corresponding to the to the scanning cycles, and to estimate based on the multiple super-resolution images at least one hemodynamic parameter of the target. In some embodiments, the recovery function includes an optimization problem selected from a list consisting of: a sparse-recovery function, a compressible-recovery function, and a regularized-recovery function.

There is additionally provided, in accordance with an embodiment of the present invention, a method for imaging including receiving a sequence of input images of a target. Each input image includes a grid of pixels representing reflections of a transmitted signal from reflectors or scatterers in the target. A resolution of the input images is degraded by a measurement process of capturing the input images in the sequence. An aggregated image, in which each pixel includes a statistical moment calculated over corresponding pixels of the input images, is derived from the sequence of input images. The aggregated image is converted into a super-resolution image of the target, having a higher resolution than the input images, by applying to the aggregated image a recovery function, which outputs the super-resolution image as a solution to the recovery function, provided that the reflectors or scatterers are sparse or compressible in the target in a predefined transform domain.

There is also provided, in accordance with an embodiment of the present invention, an apparatus for imaging including an input interface and a processor. The input interface is configured to receive a series of input images of a target. Each input image includes a grid of pixels representing reflections of a transmitted signal from reflectors or scatterers in the target. A resolution of the input images is degraded by a measurement process of capturing the input images in the series. The processor is configured to convert the input images in the series into respective temporary super-resolution images of the target, having a higher resolution than the input images, by applying to each of the input images a recovery function, which outputs the respective temporary super-resolution image as a solution of the recovery function, provided that the reflectors or scatterers are sparse or compressible in the target in a predefined transform domain, and to reconstruct an output super-resolution image of the target by aggregating the temporary super-resolution images.

There is further provided, in accordance with an embodiment of the present invention, a method for imaging including receiving a series of input images of a target. Each input image includes a grid of pixels representing reflections of a transmitted signal from reflectors or scatterers in the target. A resolution of the input images is degraded by a measurement process of capturing the input images in the series. The input images in the series are converted into respective temporary super-resolution images of the target, having a higher resolution than the input images, by applying to each of the input images a recovery function, which outputs the respective temporary super-resolution image as a solution to the recovery function, provided that the reflectors or scatterers are sparse or compressible in the target in a predefined transform domain. An output super-resolution image of the target is reconstructed by aggregating the temporary super-resolution images.

These and other embodiments will be more fully understood from the following detailed description of the embodiments thereof, taken together with the drawings in which:

### BRIEF DESCRIPTION OF THE DRAWINGS

FIG. 1 is a block diagram that schematically illustrates a block diagram of a super-resolution ultrasound imaging system, in accordance with an embodiment that is described herein;

FIG. 2 is a diagram that schematically illustrates the influence of the system Point Spread Function (PSF) on measured echoes reflected from microbubbles in blood vessels, in accordance with an embodiment that is described herein;

FIG. 3 is a diagram that schematically illustrates a sequence of IQ images acquired in an ultrasound scanning cycle, in accordance with an embodiment that is described herein; and

FIG. 4 is a flow chart that schematically illustrates a method for ultrasound super-resolution imaging, in accordance with an embodiment that is described herein.

### DETAILED DESCRIPTION OF EMBODIMENTS

### Overview

Ultrasound imaging is widely used in various diagnostic and treatment procedures. Contrast-Enhanced Ultrasound (CEUS) is an imaging method based on detecting echoes reflected from contrast agents that are injected into the circulatory system beforehand. The contrast agents typically comprise gas-filled encapsulated microbubbles. CEUS imaging enables real-time hemodynamic and perfusion imaging with high-penetration depth, but the resulting spatial resolution is typically insufficient for resolving the fine structure of the microvasculature at the capillary level.

Embodiments that are described herein provide improved methods and systems for super-resolution imaging in CEUS. Spatial resolution is inherently limited by the measurement process of capturing input images, e.g., by a Point Spread Function (PSF) of the scanning system. In principle, sub-diffraction resolution can be achieved by injecting the microbubbles to the blood stream at very low concentrations, detecting echoes of non-overlapping microbubbles, and estimating the centers of the detected non-overlapping echoes. This approach, however, typically requires acquisition times on the order of tens to hundreds of seconds, which is unsuitable for real-time, clinical imaging.

In the disclosed techniques, spatial resolution improvement is achieved by exploiting statistical properties of the received ultrasound signal and by applying sparse or compressible recovery methods.

In some embodiments, a scanning ultrasound cycle involves transmitting an ultrasound signal toward the target area and receiving a signal comprising echoes reflected from the tissue and microbubbles. The received signal is arranged in a sequence of multiple input images, wherein each input image comprises a grid of pixels representing reflections of the transmitted ultrasound signal from reflectors, scatterers or both, in the target. The visual resolution of the input images is limited by the system PSF. The reflectors and scatterers may comprise any objects in the target reflecting the transmitted ultrasound signal.

The pixels of the input images correspond to respective virtual volume cells in the target area. At any given time, a volume cell belonging to a blood vessel may contain one or more microbubbles. Moreover, due to the PSF, echoes of neighboring microbubbles may appear blurred and overlapping in the input images.

In some embodiments, the imaging system comprises a processor that processes the input images to produce a super-resolution output image. The processor first derives from the sequence of input images an aggregated image, in which each pixel comprises a statistical moment (e.g., variance) calculated over corresponding pixels of the input images. The processor then converts the aggregated image into a super-resolution image of the target, having a higher resolution than the input images, by applying to the aggregated image a predefined sparse-recovery function, or a compressible or regularized function, which resolves small blood vessels (such as arterioles, venules and the capillaries) and outputs the super-resolution image as a unique solution, provided that the reflectors or scatterers are sparse or compressible in the target in a predefined transform domain. In the super-resolution image, reflectors or scatterers corresponding to overlapping echoes appear visually separated. The method used for finding a high-resolution sparse solution is generally referred to herein as an “optimization problem.”

In some embodiments, the processor is configured to transform the aggregated image from the spatial domain to a transform domain, e.g., using a Discrete Fourier Transform (DFT), and to apply the predefined sparse-recovery function (or the compressible or regularized function) to the aggregated image in the transform domain.

In some embodiments, the aggregated image and the super-resolution image are interrelated using a model that depends on the PSF. For example, the model may comprise an underdetermined linear model, in which case the predefined sparse-recovery function comprises a convex optimization problem, which the processor solves under a sparsity constraint. The sparse recovery solution can also be found by greedy or iterative methods not based on convex optimization. In an embodiment, the optimization problem is formulated under a Total Variation (TV) constraint. In another embodiment, the optimization problem is formulated in a domain in which the solution is sparse, e.g., in terms of the locations of the microbubbles on the super-resolution grid, or under the wavelet or discrete cosine transforms.

In some embodiments, the processor separates close-by vessels by identifying different microbubbles velocities (or ranges of velocities) within the different vessels. In such embodiments, the processor decomposes the sequence of input images into multiple sequences associated with different Doppler frequency bands, produces multiple respective super-resolution images, and combines the super-resolution images to produce the output super-resolution image.

Embodiments for solving the optimization problem iteratively, which are efficient in terms of memory consumption and run time are also disclosed. Such embodiments rely on a block circulant structure of a model matrix representing the linear model, and are designed to calculate a gradient value in each iteration using Fast Fourier Transform (FFT)-based operations. Other methods for efficient implementation are also possible.

The disclosed techniques provide ultrasound imaging with both high spatial super-resolution and temporal-resolution. This is achieved mainly based on statistical independence of signal fluctuations originating from different vessels, and using sparse representation of the underlying vasculature in different dictionaries. The inventors demonstrated up to a ten-fold improvement in spatial resolution, using short scanning cycles on the order of tens of milliseconds. As such, the disclosed techniques are particularly useful in applications in which the target tissue is dynamic rather than static or in cases in which detecting fast hemodynamic changes is of interest. Example experimental and simulated results are provided in U.S. Provisional Patent Application 62/447,670, cited above.

### System Description

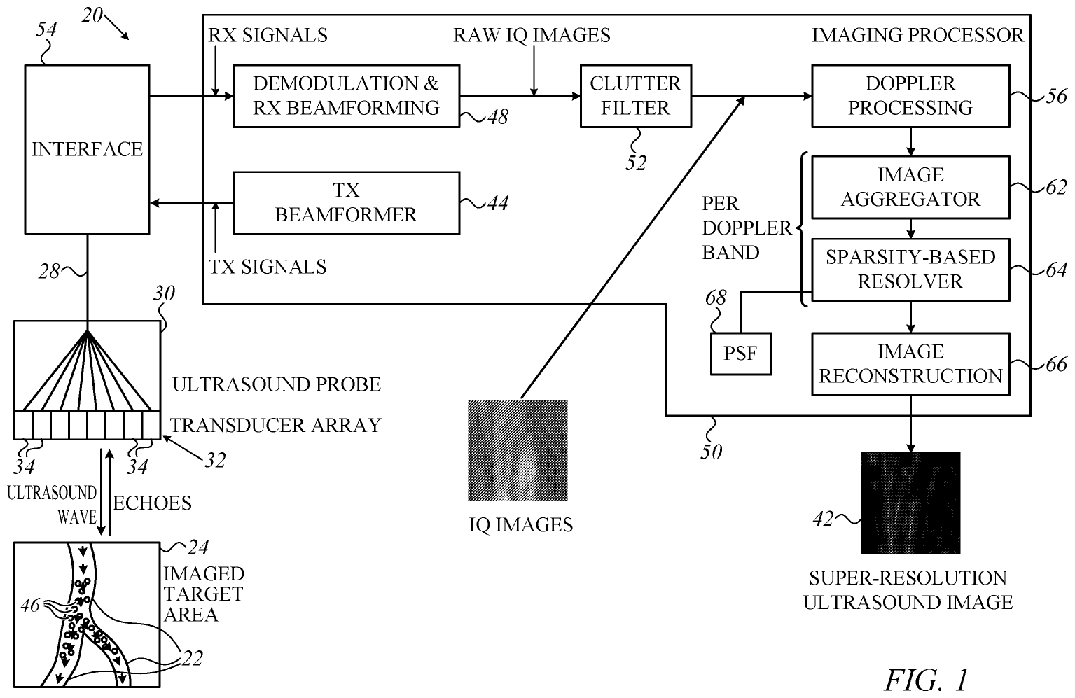

FIG. 1 is a block diagram that schematically illustrates a block diagram of a super-resolution ultrasound imaging system 20, in accordance with an embodiment that is described herein. Imaging system 20 is typically used for producing ultrasound images of a target organ of a patient. In the example of FIG. 1, system 20 serves for imaging a target area 24, which contains blood vessels 22.

Imaging system 20 comprises an ultrasound probe 30, which comprises a transducer array 32 of active elements 34. During imaging, this transducer array is typically coupled to the patient body. Transducer array 32 transmits an ultrasonic signal into the tissue, and receives respective signals that comprise reflections (“echoes”) of the transmitted signal from the tissue. In some of the disclosed techniques the transmitted signal comprises a plane wave. Alternatively, focused beams methods can also be used. The received signals are processed, using methods that are described herein, so as to reconstruct and display an ultrasound image 42 of the target organ.

In some disclosed embodiments, prior to imaging, Ultrasound Contrast Agents (UCAs) 46 are administered intravenously to the systemic circulation. UCAs 46 may comprise, for example, gas-filled encapsulated microbubbles, as is known in the art. The UCAs typically reflect ultrasound echoes much stronger than red blood cells and produce non-linear echoes. Therefore, UCAs can be used for high-contrast imaging of blood vessels structure, as well as blood perfusion in organs.

In some embodiments, the Signal to Noise Ratio (SNR) of a signal containing echoes reflected from red blood cells is sufficiently high, in which case the ultrasound imaging can be carried out without injecting the UCAs.

In the embodiment of FIG. 1, imaging system 20 comprises an imaging processor 50, which is coupled to ultrasound probe 30 via an interface 54 and a suitable link 28, which may comprise any suitable cable, typically connected at both ends, electrically and mechanically, using suitable connectors. Interface 54 exchanges TX signals and RX signals between the imaging processor and ultrasound probe 30.

In the transmit path, a TX beamformer 44 generates TX signals for controlling ultrasound wave transmission by active elements 34 of the transducer array. In some embodiments, TX beamformer 44 adjusts the amplitudes and phases of these TX signals so that active elements 34 together emit an ultrasound plane wave toward the target. In some embodiments, interface 54 comprises a Digital to Analog Converter (DAC) (not shown) for converting digital signals produced by the TX beamformer into analog signals toward the ultrasound probe. The ultrasound waves are typically transmitted as a sequence of pulses at some predefined Pulse Repetition Frequency (PRF). The transmitted pulses are modulated with a carrier signal having some predefined carrier frequency. In a typical imaging system, the PRF is on the order of 5 KHz. Depending on the depth of the imaged organ PRFs higher or lower than 5 KHz can also be used. The carrier frequency may be on the order of 4.5 MHz, depending on the active elements used and on the depth of the imaged organ. Alternatively, carrier frequencies higher or lower than 4.5 MHz can also be used. For example, carrier frequencies lower than 4.5 MHz may be used for imaging deep organs such as the heart, for imaging the brain through the skull bone and the like.

In the receive path, interface 54 receives RX signals from ultrasound probe 30, the RX signals containing echoes of the ultrasound wave reflected by the microbubbles, the blood (e.g., from red blood cells) and the surrounding tissue. Interface 54 provides the RX signals to a demodulation and RX beamforming module 48. In some embodiments, interface 54 comprises a sampler and an Analog to Digital Converter (ADC) (not shown) for converting analog signals received from the ultrasound probe into a digital form.

Demodulation and RX beamforming module 48 demodulates the RX signals based on the carrier frequency of the transmitted pulses, e.g., using quadrature sampling techniques, and applies RX beamforming to the demodulated In-phase and Quadrature (IQ) signals of the respective transducers to produce a focused image of the scanned region (target area 24). Demodulation and RX beamforming module 48 applies RX beamforming using any suitable method, e.g., by properly delaying the RX signals of respective transducers and summing the delayed signals using suitable sum-weights. In some embodiments, multi-pulse sequences are used in order to separate the non-linear (harmonic) echoes reflected from the contrast agents. These non-linear echoes are weighted and summed, by Demodulation and RX beamforming module 48, thus improving the Contrast-to-Tissue-Ratio (CTR). In an embodiment, demodulation and RX beamforming module 48 applies to the summed signal a bandpass filter (not shown) that contains the carrier frequency, which removes noise outside the passband of the transducers.

The signal output by demodulation and RX beamforming module 48 over one scanning cycle is arranged as a sequence of raw IQ images. Each raw IQ image comprises multiple complex-valued pixels, resulting from the IQ demodulation, which pixels represent the received and beamformed echoes from the UCAs.

In some embodiments a clutter filter 52 may be added to process the raw IQ images to produce a sequence of IQ images by separating between echoes reflected by microbubbles flowing in the blood stream, and the static tissue—generally referred to as clutter. In some embodiments, the clutter filter is designed with a low cutoff frequency (e.g., 0.03·PRF) in order to include in the IQ images slow flowing microbubbles while removing clutter artifacts. The output of the clutter filter is organized as a sequence of IQ images, wherein each of the IQ images comprises a two-dimensional (2D) array of complex-values pixels. The frame-rate of the IQ images is typically equal to or lower than the PRF. When using multi-pulse sequences of ultrasound pulses as noted above, the frame-rate of the IQ images is typically lower than the PRF. As another example, in some embodiments, a single IQ image is based on echoes collected over multiple PRF cycles. The sequence of IQ images is also referred to herein as an “IQ signal.”

A Doppler processing module 56 decomposes the IQ signal in accordance with blood flow velocities within the imaged vessels. In some embodiments, Doppler processing module 56 separates among the echoes in the IQ images based on the microbubbles velocities, as will be described in detail below. The output of Doppler processing module comprises multiple sequences of IQ images, each such sequence corresponds to a respective Doppler frequency or Doppler band. In some embodiments, the Doppler processing module is omitted. In such embodiments, the IQ images are processed by the image aggregator and sparsity-based resolver as equivalently belonging to a single wide Doppler band.

An image aggregator 62 accepts the sequence of IQ images for each Doppler band, and produces from the IQ images in the sequence a single aggregated image. In some embodiments, the image aggregator determines the value of each pixel in the aggregated image by calculating a suitable statistical attribute (e.g., a second order moment) over corresponding pixels of the IQ images in the sequence.

The aggregated image is further processed by a sparsity-based resolver 64, or by another suitable regularized solver, which converts the aggregated image into a super-resolution image per Doppler band. In some embodiments, the aggregated and super-resolution images are interrelated using a model that depends on the underlying PSF, and sparsity-based resolver 64 solves a convex optimization problem based on the model, under sparsity constraint, to find a unique super-resolution solution.

PSF 68 in FIG. 1 comprises the underlying system PSF, input to sparsity-based resolver 64. A sampled version of the PSF may be estimated (as will be described further below) and stored in a memory of the imaging processor. Sparsity-based resolver 64 will be described in detail further below.

An image reconstruction module 66 receives multiple sparse solutions corresponding to the Doppler bands, and reconstructs from these solutions super-resolution image 42. In some embodiments, a sparse solution comprises a super-resolution image whose pixels correspond to the structure of a sub-set of the imaged blood vessels 22, and the image reconstruction module produces output image 42 depicting the fine structure of all the blood vessels in the imaged area.

The system configuration of FIG. 1 is an example configuration, which is chosen purely for the sake of conceptual clarity. In alternative embodiments, any other suitable system configuration can be used. The elements of imaging system 20 may be implemented using hardware. Digital elements can be implemented, for example, in one or more off-the-shelf devices, Application-Specific Integrated Circuits (ASICs) or FPGAs. Analog elements can be implemented, for example, using discrete components and/or one or more analog ICs. Some system elements may be implemented, additionally or alternatively, using software running on a suitable processor, e.g., a Digital Signal Processor (DSP). Some system elements may be implemented using a combination of hardware and software elements.

In some embodiments, some or all of the functions of imaging system 20 may be implemented using a general-purpose processor, which is programmed in software to carry out the functions described herein. The software may be downloaded to the processor in electronic form, over a network, for example, or it may, alternatively or additionally, be provided and/or stored on non-transitory tangible media, such as magnetic, optical, or electronic memory.

System elements that are not mandatory for understanding of the disclosed techniques, such as circuitry elements relating to interface 54, are omitted for the sake of clarity or only briefly discussed.

### Signal Model and Doppler Analysis for Vascular Imaging

In a scanning cycle for producing a single super-resolution image 42, imaging system 20 transmits, using transducer array 34, a series of ultrasound pulses toward the target region of interest 24. The ultrasound pluses are typically transmitted at a time-interval ΔT=1/PRF. Echoes created by the transmitted ultrasound pulses reflected from tissue, blood and microbubbles, are detected by the transducers, which produce respective RX signals. The RX signals are sampled, and processed using demodulation and RX beamforming module 48 as described above, to produce the sequence of raw IQ images, also referred to herein as a raw IQ signal.

Over the scanning cycle interval t∈[0, T], during which multiple images are typically acquired, the raw IQ signal is given by:

ƒ(x,z,t)=c(x,z,t)+b(x,z,t)+w(x,z,t), 0≤t≤T Equation 1:

wherein b denotes the desired signal containing echoes reflected from the microbubbles, c denotes clutter echoes reflected by tissue. The signal w denotes an additive noise signal that can be modeled as an independent and identically distributed (iid) thermal noise. The desired signal b is also referred to herein as a “blood signal.” The variables x and z denote lateral and axial coordinates, respectively. Based on Equation 1, the imaging processor produces an estimated signal \hat{b} of the desired signal b given the raw IQ signal ƒ.

Removing the clutter signal c from the raw IQ signal ƒ is based on the following assumptions. Firstly, at low acoustic pressures (such as normally used in ultrasound) the echoes reflected by the microbubbles have a non-linear nature (i.e., harmonic), in contrast to essentially linear echoes caused by tissue. In some embodiments, the pulses transmitted are modulated with specially designed amplitudes and/or phases, so as to emphasize this non-linearity. In such embodiments, using pulse modulation, e.g., Pulse Inversion (PI) techniques, enables to reject linear echoes (tissue) while retaining non-linear second harmonic echoes from the microbubbles. Alternatively or additionally, methods such as amplitude modulation (AM) and combinations of PI and AM, also referred to as AMPI, can also be used. Secondly, during the acquiring cycle, the tissue is assumed to be stationary or slowly moving, whereas the microbubbles flow in the blood stream at higher speeds. As a result, tissue clutter and the microbubbles produce distinguishable velocity patterns.

In some embodiments, based on the above assumptions, clutter filter 52 is designed to apply to the raw IQ signal temporal or spatiotemporal filtering techniques to produce the clutter-free IQ signal. In an embodiment, clutter filter 52 applies to the raw IQ signal IIR filters with projection initialization. In such embodiments, the cutoff parameter of the IIR filter relates to the minimal expected velocity of the microbubbles in the blood stream. Clutter filtering methods such as those mentioned above are described, for example, by Bjaerum et al. in “Clutter filter design for ultrasound colour flow imaging,” IEEE Transactions on Ultrasonic, Ferroelectric and Frequency Control,” volume 49, number 2, 2002, pages 204-209, and by Demené et al. in “Spatiotemporal Clutter Filtering of Ultrafast Ultrasound Data Highly Increases Doppler and fUltrasound Sensitivity,” IEEE Transactions on Medical Imaging, volume 34, number 11, 2015, pages 2271-2285.

In alternative embodiments, instead of IIR filtering, clutter filter 52 removes undesired clutter from the signal using singular-value decomposition (SVD) techniques.

The IQ signal b comprises echoes reflected from a time-dependent set K(t) of microbubbles, wherein (xₖ(t), zₖ(t)) denotes the time-dependent position of the k^{th} microbubble. The echoes reflected from the microbubbles in the set K(t) contribute to the signal \hat{b} at a position (x, z) as given by:

$$\hat{b} ( x , z , t ) = {\sum }_{k ∈ K ( t )} H ( x - {x}_{k} ( t ) , z - {z}_{k} ( t ) ) \cdot {σ}_{k} \qquad Equation 2$$

wherein H(x,z) denotes the Point Spread Function (PSF) of the imaging system, and σₖ denotes the scattering cross-section of the k^{th} microbubble. As seen in Equation 2, the contribution of each microbubble to the signal depends on the underlying PSF, the distance of the microbubble from the measured point and the scattering cross-section σₖ associated with that microbubble.

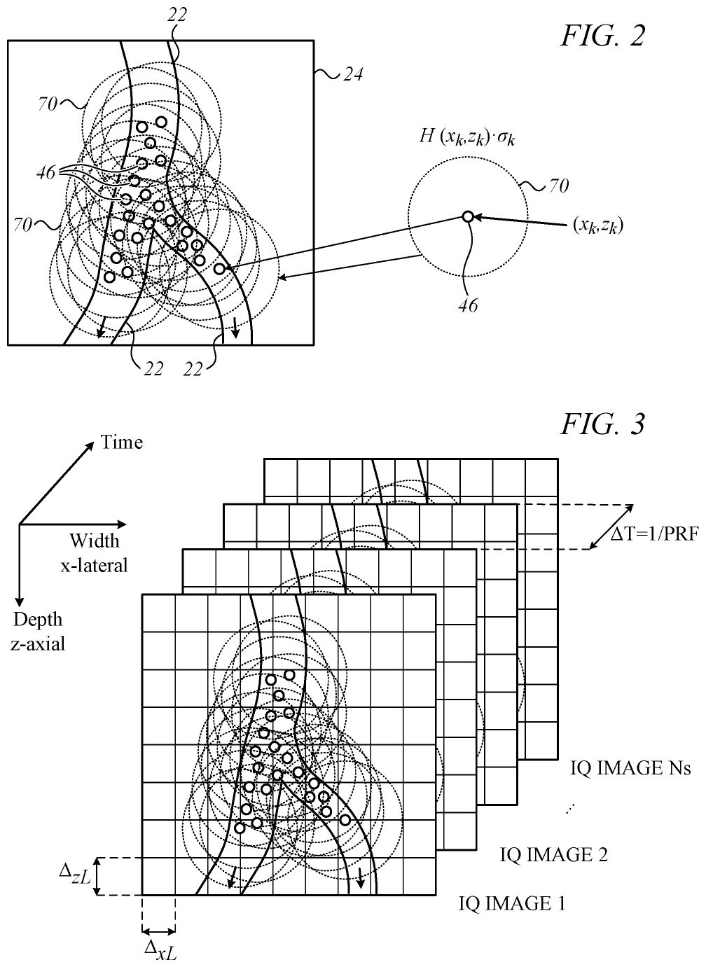

FIG. 2 is a diagram that schematically illustrates the influence of the system Point Spread Function (PSF) on measured echoes reflected from microbubbles in blood vessels, in accordance with an embodiment that is described herein. The figure depicts microbubbles 46 of the set K(t) that fall within the measured area 24.

In the present example, the PSF function H(x, z) blurs point microbubbles 46 to appear in the IQ image as circles 70. Alternatively, other suitable PSFs having blur-patterns other than a circle can also be used. For example, in some practical implementations, the PSF has an oval shape whose size along the axial axis is larger than its size along the lateral axis. The amplitude of the PSF about its center is typically approximated as a modulated Gaussian.

As seen in the figure, circles 70 of neighboring microbubbles may overlap. In case of overlap, the corresponding microbubbles cannot be separately resolvable. As a result, the visual resolution is limited by the PSF, and the splitting of blood vessel 22 cannot be directly imaged.

In practice, the beamformed signal is typically spatially quantized in a grid of low-resolution pixels. Consider a grid comprising low-resolution pixels of size (Δ_{L}, Δ_{zL}) Let Np denote the number of pixels in the grid that contain at least one microbubble, and let (xₙ, zₙ), n=1 . . . Np denote the discrete center positions of these pixels on the grid. We assume that Np does not change over the scanning cycle period. The IQ signal at quantized locations x→mΔ_{xL} and z→lΔ_{zL} can be approximated as given by:

$$\hat{b} ( m {Δ}_{xL} , l {Δ}_{zL} , t ) \approx {\sum }_{n = 1}^{Np} H ( m {Δ}_{xL} - {x}_{n} , l {Δ}_{zL} - {z}_{n} ) \cdot {S}_{n} ( t ) \qquad Equation 3$$

In Equation 3, Sₙ(t) is a time-dependent fluctuation function of the n^{th} pixel that will be described in detail below.

FIG. 3 is a diagram that schematically illustrates a sequence of IQ images acquired in an ultrasound scanning cycle, in accordance with an embodiment that is described herein. In the present example, the scanning cycle in FIG. 3 contains Ns IQ images separated by time intervals ΔT=1/PRF. Alternatively, frame-rates other than the PRF can also be used. The series of IQ images is also referred to herein as a “movie.”

As noted above, an IQ image comprises a grid of low-resolution pixels having lateral and axial sizes Δ_{xL} and Δ_{zL}, respectively. The IQ image at time pΔT corresponds to the complex-valued measurements of the signal \hat{b}(mΔ_{xL}, lΔ_{zL}, pΔT) over the pixel indices 0≤m≤Mx and 0≤l≤Mz. In the example of FIG. 3, Mx=9 and Mz=8, i.e., a total number of 72 pixels. In practical implementations, however, IQ images having other suitable number of pixels, e.g., an IQ image of 512-by-1640 pixels can also be used. The actual resolution selected for the IQ images may depend, for example, on the specific organ being imaged.

The per-pixel fluctuation function Sₙ(t) in Equation 3 above can be modeled as a complex-valued function comprising a multiplicative envelope component α(t) and a time-dependent complex phase component. The phase component of the fluctuation function changes over consecutive acquisition periods ΔT depending on the velocity of the microbubbles. Statistical properties of the fluctuation function that are exploited for enhancing spatial resolution while maintaining high temporal resolution will be described further below.

Let p denote the index of the transmitted ultrasound pulse. At a time instance pΔT, the fluctuation function is thus given by:

$${S}_{n} ( p Δ T ) = a [ p Δ T ] \cdot {\sum }_{u ∈ {U}_{n}} exp ( - j \cdot {v}_{n} \cdot p Δ T + {β}_{0} ) \qquad Equation 4$$

wherein Uₙ is a set of microbubble-velocities detected within the n^{th} pixel, vᵤ is the Doppler frequency corresponding to the microbubble-velocity u∈Uₙ, and β₀ is a random phase. The Doppler frequency vᵤ is related to an axial velocity v_{u,z} as given by:

$${v}_{u} ≡ \frac{4 π {f}_{0} {v}_{u , z}}{C} \qquad Equation 5$$

wherein C denotes the velocity of sound in the medium, and ƒ₀ denotes the center frequency of the ultrasound wave (the carrier frequency of the transmitted ultrasound pulses).

### Doppler Analysis

In some embodiments, Doppler processing module 56 separates the sequence of IQ images, into multiple sequences corresponding to respective or Doppler frequencies.

Let Aᵤ[k], k=0 . . . P−1 denote coefficients of a P-point Discrete Fourier Transform (DFT) applied to a series of samples Sₙ(pΔT) of the fluctuation function corresponding to Doppler frequency vᵤ. Using Equation 4, the DFT coefficients Aᵤ[k] are given by:

Aᵤ[k]=DFT{a[pΔT]·exp(−j·vᵤ·pΔT+β₀)}[k], k=0 . . . P−1 Equation 6:

wherein the P-point DFT over a series {x[n]}, n=0 . . . N−1 is defined as:

$$DFT \{ x [ n ] \} [ k ] = {\sum }_{p = 0}^{N - 1} x [ n ] \cdot exp ( - \frac{j 2 π kn}{N} ) , \\ k = 0 … P - 1 \qquad Equation 7$$

Applying a temporal P-point DFT to the signal of Equation 3, sampled at times pΔT, for each pixel [m, l] in the low-resolution grid results in the DFT coefficients B as follows:

$$B [ k \vert m {Δ}_{xL} , l {Δ}_{zL} ] = DFT \{ {\sum }_{n = 1}^{Np} H ( m {Δ}_{xL} - {x}_{n} , l {Δ}_{zL} - {z}_{n} ) \cdot {S}_{n} ( p Δ T ) \} = {\sum }_{n = 1}^{Np} {\sum }_{u ∈ {U}_{n}} H ( m {Δ}_{xL} - {x}_{n} , l {Δ}_{zL} - {z}_{n} ) \cdot {A}_{u} [ k ] \qquad Equation 8$$

wherein B[k|mΔ_{xL}, lΔ_{zL}], k=1 . . . P−1 are the DFT coefficients of the transformed IQ signal. Using the number U of microbubble-velocities over the entire scanning cycle, and defining Nu as the number of microbubbles of u^{th} microbubble-velocity, Equation 8 can be rewritten as:

$$B [ k \vert m {Δ}_{xL} , l {Δ}_{zL} ] = {\sum }_{u ∈ U} ( {A}_{u} [ k ] \cdot {\sum }_{n = 1}^{Nu} H ( m {Δ}_{xL} - {x}_{n} , l {Δ}_{zL} - {z}_{n} ) ) \qquad Equation 9$$

In Equation 9, the signal is spectrally decomposed according to the Doppler frequencies vᵤ, u∈U.

In some embodiments, Doppler processing module 56 decomposes the IQ signal into multiple Doppler bands, whose total number is denoted D_{ƒ}. Let h_{d}(pΔT) be the impulse response function of a bandpass filter for the d^{th} Doppler band, d=1 . . . D_{ƒ}. A Doppler band typically corresponds to a respective range of microbubble-velocities. In the transform domain, the bandpass filter is defined by the DFT coefficients H_{d}[k], k=0 . . . P−1 of a P-point DFT applied to h_{d}(pΔT). Based on the convolution theorem, the IQ signal can be filtered by calculating a scalar product between a first vector B[k|mΔ_{xL}, lΔ_{zL}] containing the DFT coefficients of the IQ signal and a second vector containing the DFT coefficients H_{d}[k] of the filter impulse response, and applying an inverse DFT transform as given by:

\hat{b}_{d}[pΔT|mΔ_{xL},lΔ_{zL}]=DFT⁻¹{B[k|mΔ_{xL},lΔ_{zL}]·H_{d}[k]}ₖ₌₀^{P-1} Equation 10:

The inverse DFT transform is given by:

$${DFT}^{- 1} \{ X [ k ] \} [ n ] = \frac{1}{P} {\sum }_{k = 0}^{P - 1} X [ k ] \cdot exp ( \frac{j 2 π kn}{P} ) , \\ n = 0 … N - 1 \qquad Equation 11$$

In some embodiments, the signal \hat{b}_{d} of Equation 10 comprises a movie of images structured similarly to the IQ images of FIG. 3. The different D_{ƒ} movies typically correspond to different flow patterns, and therefore can be used to identify fine vasculature structure. For example, Doppler analysis can be used to distinguish between positive and negative Doppler frequencies, corresponding to blood flow in atrial or venous vasculature.

### Statistical Properties of the IQ Signal

As noted above, statistical properties of fluctuation function can be used for enhancing imaging spatial resolution. The target area can be viewed as partitioned into virtual volume cells corresponding to the pixels in the IQ images. At any time instance, each volume cell in the target area contains one or more microbubbles, or alternatively contains no microbubbles at all. The actual number of microbubbles contained in a volume cell depends on the structure of the vasculature and on the blood flow patterns within the imaged vessels. Next we present two assumptions regarding the statistical properties of the fluctuation function, although other assumptions are possible as well:

A1. For a processed ensemble, e.g., the IQ signal comprising Ns IQ images of the scanning cycle, Sₙ(t) is considered a wide sense stationary process. This means that the statistical properties of the fluctuation function remain unchanged during the scanning cycle. Therefore, the location of a vessel containing volume cells positioned around (xₙ, zₙ), n=1 . . . Np, does not change during the acquisition time of the processed ensemble. In an embodiment, in case of extreme motion artifacts this assumption may facilitate in applying image registration. This assumption is not mandatory for the disclosed techniques, and is made for simplicity.

A2. Temporal fluctuations in volume cells corresponding to different blood vessels are statistically independent. Specifically, let Sₙ₁(t) and Sₙ₂(t) be fluctuation functions in respective volume cells n1 and n2 of two different blood vessels. When the flow patterns (microbubble velocities and directions) in the two blood vessels are different, Sₙ₁(t) and Sₙ₂(t) can be considers statistically independent, as given by:

E{\tilde{S}ₙ₁(t1)\overline{\tilde{S}ₙ₂(t2)}}=0, ∀n1≠n2 Equation 12:

wherein Śₙ(t)=Sₙ(t)−E{Sₙ(t)}.

Based on the statistical assumptions above, an aggregated image G₂ (r, τ) comprising, e.g., an autocorrelation function of the IQ signal at a point r=(x, z) for a predefined delay τ can be defined as follows:

$${G}_{2} ( r , τ ) = {\sum }_{n} {\vert H \vert}^{2} ( r - {r}_{n} ) \cdot E \{ {\tilde{S}}_{n} ( t + τ ) \overline{{\tilde{S}}_{n} ( t )} \} + {{\sum }_{i , j}}_{i \neq j} H ( r - {r}_{i} ) \cdot \overline{H} ( r - {r}_{j} ) \cdot E \{ {\tilde{S}}_{i} ( t + τ ) \overline{{\tilde{S}}_{J} ( t )} \} \qquad Equation 13$$

wherein rₙ=(xₙ, zₙ), and the index n runs over the entire pixels of the IQ image. The indices i, j correspond to volume cells located in the same blood vessel or streamline and are therefore assumed to be correlated.

In Equation 13, the term E{\tilde{S}ₙ(t+τ)\overline{\tilde{S}ₙ(t)}} in the first sum is the autocorrelation function of the temporal intensity fluctuations of pixel n, and the term E{\tilde{S}ᵢ(t+τ)\overline{\tilde{S}ⱼ(t)}} refers to the cross-correlation of the temporal intensity fluctuations between pixels i and j.

Although in Equation 13, the aggregated image comprises second order moments, other suitable statistical attributes such as moments of higher order can also be used.

In Equation 13, G₂ (r, τ) is expressed by a first sum, corresponding to microbubbles flowing in different vessels, and a second sum, corresponding to flows within the same vessel. Note that cross terms corresponding to volume cells belonging to different blood vessels are omitted from the first sum because these cross terms cancel out under the second assumption above.

The first sum represents the improved separation between microbubbles flowing independently in different vessels. The second sum creates a smoothing effect, affecting microbubbles flowing along the same vessel. Since the second sum smoothens the aggregated image in the direction of the vessels, but essentially does not change the shape of the underlying vasculature, this sum can be considered as an additive noise component.

Using an aggregated image rather than the IQ images, improves the Signal to Noise Ratio (SNR) because the aggregation operation emphasizes correlated signals relative to the thermal noise. In addition, aggregation using second order statistics improves spatial resolution, by a factor on the order of \sqrt{2}, compared to pixel-wise averaging of the IQ images. Higher order statistics typically require a large number of IQ images in a scanning cycle, which is undesirable, e.g., for real-time applications. Moreover, the phenomenon of strong echoes masking week echoes typically worsens with high-order statistics.

Next we describe embodiments for converting the low-resolution aggregated image into a super-resolution image using sparse recovery techniques, or compressible or regularized techniques.

### Sparse Recovery for Achieving Spatial Super-Resolution

For achieving super-resolution imaging, the underlying vasculature is modeled as point-targets on a super-resolution grid that is much finer than the low-resolution grid used for IQ images and the aggregated image. We further assume that over the super-resolution grid the underlying vasculature is compressible in some domain, or sparse, and therefore can be estimated using sparse recovery techniques in a suitable domain.

The goal is to identify a set of pixels on the super-resolution grid that contain blood vessels. Using sparse recovery methods, the super-resolution image of the vasculature can be derived from an aggregated image calculated from IQ images having overlapping echoes.

Let [Δ_{xH}, Δ_{zH}] denote the pixel size of the super-resolution grid. We assume that the pixel size of the low-resolution grid is D times larger than the pixel-size of the super-resolution grid, in each dimension:

[Δ_{xL},Δ_{zL}]=[DΔ_{xH},DΔ_{zH}], D>1 Equation 14:

A point [xₙ, zₙ] on the super-resolution grid can be expressed as [xₙ, zₙ]=[iₓΔ_{xH}, i_{z}Δ_{zH}] for some iₓ, i_{z}∈{0 . . . N−1}. Using these notations, an M-by-M aggregated image will be converted to an N-by-N super-resolution image, wherein N=D·M, and D>1.

By omitting the noisy term (i.e., the second sum) in Equation 13 as explained above, G₂ of Equation 13 can be approximated as:

$${G}_{2} ( m {Δ}_{xL} , l {Δ}_{zL} , τ ) = {\sum }_{{i}_{x} , {i}_{z} = 0}^{N - 1} {\vert H \vert}^{2} ( m {Δ}_{xL} - {i}_{x} {Δ}_{xH} , l {Δ}_{zL} - {i}_{z} {Δ}_{zH} ) \cdot {J}_{{i}_{x} , {i}_{z}} ( τ ) \qquad Equation 15$$

wherein Jᵢ_{ₓ}_{,i}_{_{z}}(τ) represents the autocorrelation of pixel [iₓ, i_{z}] on the super-resolution grid, for a pre-chosen time-lag r as given by:

Jᵢ_{ₓ}_{,i}_{_{z}}(τ)=E{\tilde{S}ᵢ_{ₓ}(t+τ)\overline{\tilde{S}ₗ_{_{z}}(t)}} Equation 16:

By estimating the locations [iₓΔ_{xH}, i_{z}Δ_{zH}] on the super-resolution grid, for which Jᵢ_{ₓ}_{,i}_{_{z}}(τ)≠0, the vascular structure can estimated at the super-resolution level. Note that the autocorrelation of a pixel that contains no microbubbles is zero for any selected time-lag.

In some embodiments, the aggregated image, which is represented by G₂(·) in Equation 15, is calculated for a zero time-lag, i.e., τ=0. In such embodiments, the variances of the fluctuations are estimated on the super-resolution grid. Each pixel in the recovered super-resolution image corresponds to the variance of the echoes originating from corresponding volume cells (or zero, if no echoes are detected).

In some embodiments, estimating the vasculature over the super-resolution grid is carried out efficiently in the frequency domain, as described herein. Using the notations Δ_{xL}=DΔ_{xH} and Δ_{zL}=DΔ_{zH}, and omitting Δ_{xL}, Δ_{zL}, Δ_{xH}, Δ_{zH} for simplicity, Equation 15 can be written as:

$$Equation 17$$

$${G}_{2} ( mD , lD , τ ) = {\sum }_{{i}_{x} , {i}_{z} = 0}^{N - 1} {\vert H \vert}^{2} ( mD - {i}_{x} , lD - {i}_{z} ) \cdot {J}_{{i}_{x} , {i}_{z}} ( τ )$$

wherein G₂ (mD, lD, τ) is an M-by-M matrix. A formulation of this sort is described, for example, by Solomon et al. in “Sparsity-based Ultrasound Super-resolution Hemodynamic Imaging,” arXiv preprint, Dec. 2, 2017, arXiv:1712.00648, which is incorporated herein by reference.

Let G₂^{F}[kₘ, kₗ, τ] denote an M-by-M Two-Dimensional Discrete Fourier Transform (2D-DFT) of G₂ (mD, lD, τ) given by:

$$Equation 18 \\ {G}_{2}^{F} [ {k}_{m} , {k}_{l} , τ ] = {U}^{F} [ {k}_{m} , {k}_{l} ] {\sum }_{{i}_{x} , {i}_{z} = 0}^{N - 1} {J}_{{i}_{x} , {i}_{z}} ( τ ) {e}^{- j \frac{2 π}{N} {k}_{m} {i}_{x}} {e}^{- j \frac{2 π}{N} {k}_{l} {i}_{z}} \qquad$$

wherein U^{F}[kₘ, kₗ], kₘ, kₗ=0 M−1 is an M-by-M 2D-DFT of the M-by-M squared absolute values PSF function |H|² (xD,yD). The indices kₘ, kₗ=0 . . . M−1 are indices of respective spatial frequencies.

For using sparse recovery formulation, the M columns of G₂^{F}[kₘ, kₗ, τ] are stacked to produce a M² long vector denoted γ(τ), and the N columns of Jᵢ_{ₓ}_{,i}_{_{z}}(τ) are stacked to produce a N² long vector x(τ). The vector x(τ) represents the underlying vasculature that needs to be recovered on the super-resolution grid, and is therefore assumed to be sparse or compressible in an appropriate basis. Using the above vector notations, Equation 18 is rewritten in a matrix-vector form as:

y(τ)=W(F_{M}⊗F_{M})x(τ)=Ax(τ), A∈ [symbol] ^{M}^{²}^{×N}^{²} Equation 19:

In Equation 19, A=W(F_{M}⊗F_{M}), wherein W is a M²-by-M² diagonal matrix whose M² diagonal elements are {U^{F}[0,0] . . . U^{F}[M−1, M−1]} of the 2D-DFT of the PSF. For example, the first M elements on the diagonal of W correspond to the first column of U^{F}, the next M elements on the diagonal of W correspond to the second column of U^{F}, and so on.

In Equation 19, F_{M} is a partial M-by-N DFT matrix, created by taking the M rows of a full N×N DFT matrix corresponding to the lowest M frequency components. For example, let F_{N} denote a full N-by-N DFT matrix whose spatial frequency indices run between −N/2+1 and N/2, F_{M} can be constructed by taking the M rows of F_{N} whose frequency indices run between −M/2+1 and M/2. The elements of the matrix F_{N} have the form:

$${F}_{N} [ k , l ] = {e}^{- j \frac{2 π}{N} kl}$$

The symbol ⊗ in Equation 19 denotes the Kronecker product operator. In some embodiments, the matrix A can be calculated only once and stored in memory at initialization. The matrix A can be calculated efficiently using Fast Fourier Transform (FFT) operations. In alternative embodiments, data structures much smaller than A are calculated and stored. In these embodiments, although A is not used explicitly, matrix-by-vector multiplication of the form Ax can be calculated efficiently based on the pre-calculated small data structures, using FFT-based operations. Embodiments that solve a sparse-recovery problem efficiently, for producing the super-resolution image, using FFT operations, without explicitly using the matrix A will be described further below.

The vasculature structure represented by x(r) can be resolved by assuming a model y=Ax+ω, wherein y=y(τ), x=x(τ), and ω is additive noise. This is an underdetermined linear model that typically has multiple solutions. A convex optimization problem for resolving the vasculature is given by:

$$\hat{x} = {min}_{x} \{ λ {‖ x ‖}_{1} + \frac{1}{2} {‖ y - Ax ‖}_{2}^{2} \} \qquad Equation 20$$

wherein λ≥0 is a regularization parameter. In Equation 20, y is a measurement vector and x is the unknown super-resolution sparse vector to be resolved. Other suitable methods for solving y=Ax+ω for x are possible using more general sparse recovery methods or compressible methods in appropriate domains can also be used.

In some embodiments, to find a unique solution, the convex optimization problem in Equation 20 is solved under a sparsity constraint, i.e., the solution 2 is sparse. In some embodiments, the convex optimization problem is solved iteratively, by sparsity-based resolver 64, to converge to the global minimum solution, or a solution sufficiently close to this global minimum. In alternative embodiments, other suitable sparse recovery solvers are used, which also allow sparsity and compressibility in other domains.

When preselecting a zero time-lag, calculating Jᵢ_{ₓ}_{,i}_{_{z}}(τ=0) in Equation 16 refers to pixel-wise variances, which are non-negative. In this case, sparsity-based resolver 64 resolves the optimization problem under an additional constraint x≥0, which allows fast convergence to the global minimum solution.

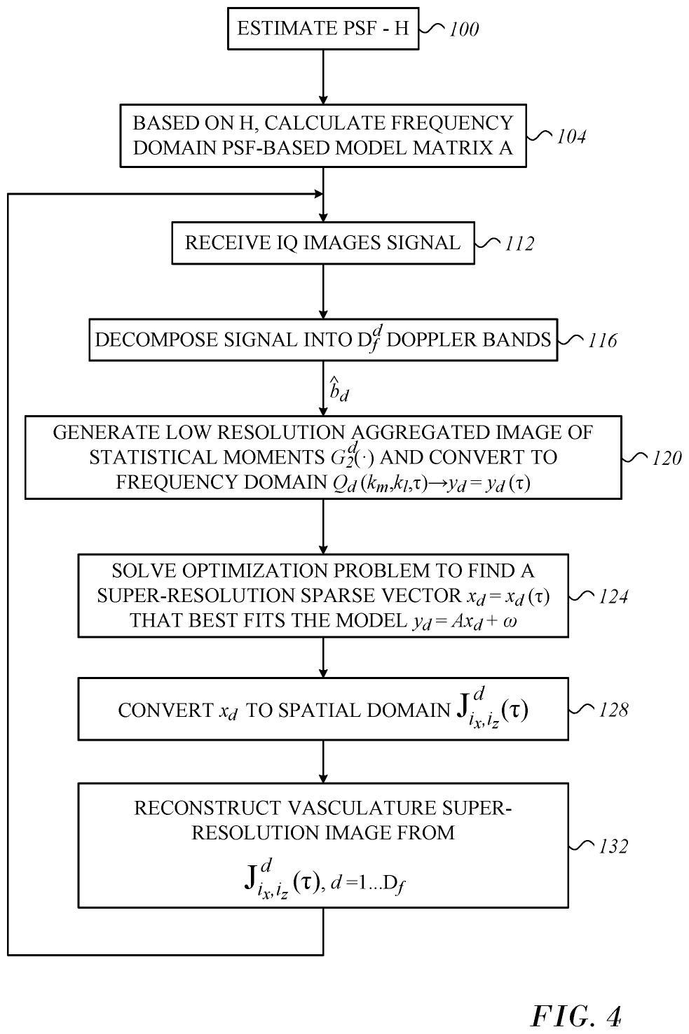

FIG. 4 is a flow chart that schematically illustrates a method for ultrasound super-resolution imaging, in accordance with an embodiment that is described herein. In describing the method, we assume that multiple echoes of transmitted ultrasound pulses have been collected during a scanning cycle, sampled and processed by demodulation and RX beamforming module 48 and clutter filter 52 of imaging processor 50, to produce multiple IQ images.

The method begins with imaging processor 50 estimating the PSF of the overall imaging system including ultrasound probe 30, at a PSF estimation step 100. In the present example, estimating the PSF is based on identifying echoes from resolvable microbubbles as follows. First, the imaging processor calculates multiple correlations between each of multiple respective M-by-M image patches and an M-by-M template patch. In some embodiments, the template patch and/or image patches are acquired for the purpose of PSF estimation. In other embodiments, PSF estimation is based on the IQ frames acquired for imaging as will be described at step 112 below. The template patch can be picked manually, for example, or computed based on the geometry of the transducers and the imaging depth. Assume the imaging processor identifies a number L1 of patch images whose correlation with the template patch is above a predefined correlation-threshold. In an embodiment, the imaging processor aligns the L1 patch images to the template patch using rigid body registration methods, and estimates the M-by-M matrix H of the PSF by taking the mean of each pixel over the L1 aligned patch images.

The methods described above for estimating the PSF are not mandatory, and any other suitable method for PSF estimation can also be used. In alternative embodiments, the PSF is provided to imaging processor 50 via a suitable interface (not shown) in which case the method may skip step 100. In yet another embodiment, the PSF is not pre-estimated but is rather solved for together with the super-resolution image x.

At a modeling step 104, the imaging processor uses the estimated PSF function H, to calculate a PSF-based model represented by an M²-by-M² matrix A of Equation 19. To this end, the imaging processor calculates an M-by-M matrix U^{F} [kₘ, k₁], kₘ, k₁=0 . . . M−1 by applying a 2D-DFT to M² samples of the PSF function, and calculates the model matrix A as:

A=Diag{U^{F}[0,0], . . . , U^{F}[M−1,M−1]}(F_{M}⊗F_{M}) Equation 21:

Alternative embodiments that do not use A explicitly, and are therefore efficient in terms of storage space and execution time will be described in detail below.

At a signal reception step 112, Doppler processing module 56 receives an IQ signal \hat{b}(mΔ_{xL}, lΔ_{zL}, t) as given in Equation 3. The IQ signal is arranged as a sequence of Ns IQ images of M-by-M pixels.

(The term “resolution” should not be confused with the term “visual resolution.” In the present context, the term “resolution” refers to the pixel size of the image, after beamforming, regardless of the channel or the image acquisition system. The term “visual resolution” refers to the actual optical sharpness or blurriness of the image, e.g., due to the image acquisition system and/or the channel, regardless of the pixel sizes of the image.) As such, the IQ signals are sampled on a grid that complements the low resolution dictated by the PSF, to produce the IQ images.

At a decomposition step 116, the Doppler processing module decomposes the IQ signal \hat{b} into multiple Doppler bands:

\hat{b}_{d}(mΔ_{xL},lΔ_{zL},t), d=1 . . . D_{ƒ}, m,l=0 . . . M−1 Equation 22:

Each of the decomposed signals \hat{b}_{d} comprises a respective movie comprising Ns M-by-M IQ images. Doppler processing in the frequency domain is described, for example, in Equations 6-11 above.

At an autocorrelation step 120, image aggregator 62 calculates for each Doppler band an M-by-M aggregated image, e.g., a pixel-wise autocorrelation function over the IQ images of the relevant Doppler band to produce for each Doppler band a respective aggregated image G₂^{d}(·). Alternatively, statistical moments having order higher than 2 can also be used. In an embodiment, the image aggregator calculates the aggregated image by estimating empirically, from the Doppler filtered signal, G₂^{d}(m, l, τ)=E{\tilde{b}_{d}(m, l, t)\overline{\tilde{b}_{d}(M, l, t+τ)}}, wherein \tilde{b}_{d}(m, l, t)=\hat{b}_{d}(m, l, t)−E{\hat{b}_{d}(m, l, t)}. Further at step 120, the image aggregator transforms the aggregated image G₂^{d}(·) into the frequency domain using a 2D-DFT, and stacks the columns of the resulting transformed matrix, denoted Q_{d}(kₘ, kₗ, τ), to produce a vector y_{d}=y_{d}(τ) of length M².

At an optimization problem solving step 124, sparsity-based resolver 64 assumes a model:

y_{d}=Ax_{d}+ω Equation 23:

The example model in Equation 23 is a linear model that relates between the (low resolution) measurements y_{d}=y_{d}(τ) and the desired (super-resolution) vector x_{d}=x_{d}(τ), wherein ω is an additive noise vector.

Finding a sparse vector x_{d} that best fits the model can be carried out in various ways. In some embodiments, sparsity-based resolver 64 estimates x_{d} by solving the optimization problem of Equation 20, applied to the relevant Doppler band:

$${\hat{x}}_{d} = {min}_{{x}_{d}} \{ λ {‖ {x}_{d} ‖}_{1} + \frac{1}{2} {‖ {y}_{d} - {Ax}_{d} ‖}_{2}^{2} \} \qquad Equation 24$$

Equation 24 can be solved, for example, using the methods described by Beck and Teboulle in, “A Fast Iterative Shrinkage-Thresholding Algorithm,” SIAM Journal on Imaging Sciences, volume 2, number 1, 2009, pages 183-202.

In the embodiments described above, the optimization problem was formulated in the frequency domain, i.e., A and y_{d} are derived using suitable DFTs. This representation, however, is not mandatory, and other suitable domains or dictionaries for sparse representation such as Discrete Cosine Transform (DCT), Haar wavelets or Daubechies wavelets can also be used.

In some embodiments, a model similar to the model Equation 23 can be resolved under Total-Variation (TV) constraint. In the TV framework the optimization problem is given by:

$${\hat{x}}_{d} = {min}_{{x}_{d}} \{ λ {TV ( {x}_{d} )}_{1} + \frac{1}{2} {‖ {y}_{d} - {Ax}_{d} ‖}_{2}^{2} \} \qquad Equation 25$$

The TV constraint in Equation 25 can be isotropic, i.e., uniform in all directions, or anisotropic. Equation 25 can be solved, for example, using the methods described by Beck and Teboulle in “Fast gradient-based algorithms for constrained total variation image denoising and deblurring problems,” IEEE Transactions of Image Processing, volume 18, number 11, 2009, pages 2419-2434. Other techniques based on non-convex optimization can also be applied. Such methods include (but are not limited to) greedy methods and majorization-minimization methods (e.g., reweighted 11).

In alternative embodiments, sparsity may also be assumed using dictionaries such as wavelet or the Discrete Cosine Transform (DCT). A general formulation of this sort may be given as:

$${\hat{x}}_{d} = {min}_{{x}_{d}} \{ λ {‖ {T}^{*} {x}_{d} ‖}_{1} + \frac{1}{2} {‖ {y}_{d} - {Ax}_{d} ‖}_{2}^{2} \} \qquad Equation 26$$

wherein T comprises the underlying transformation (e.g., wavelet or DCT) in matrix form, and the operator (·)* denotes the Adjoint operator (e.g., a complex conjugate operator in case of complex numbers.) Solving Equation 26 under a sparsity constraint results in a vector x_{d} that is sparse in the underlying basis of T. Equation 26 can be solved, for example, using the methods described by Tan et al. in “Smoothing and decomposition for analysis sparse recovery,” IEEE Transactions of Signal Processing, volume 62, number 7, 2014, pages 1762-1774.

At a conversion to spatial domain step 128, image reconstruction module 66 converts \hat{x}_{d} (of length N²) estimated at step 124 back to the spatial domain, by re-ordering \hat{x}_{d} to produce the N-by-N spatial domain image Jᵢ_{ₓ}_{,i}_{_{z}}^{d}(τ). In an embodiment, the reconstruction module additionally convolves the constructed image with a small-sized kernel having an impulse response of a low-pass filter, to produce a visually smooth image. Such a kernel may comprise, for example, PSF 68 on the low resolution grid.

At a reconstruction step 132, the image reconstruction module 66 constructs a final super-resolution image, from the multiple spatial domain images recovered at step 128 for the multiple Doppler bands. In some embodiments, the image reconstruction module identifies the non-zero pixels of one or more Jᵢ_{ₓ}_{,i}_{_{z}}^{d}(τ) as the interior of corresponding blood vessels, and reconstructs the blood-vessels, accordingly. In some embodiments, the image reconstruction module assigns different colors to blood vessels reconstructed from different Doppler bands.

Following step 132 the method loops back to step 132 to receive subsequent IQ images.

In some embodiments, multiple sequences of the input images are received over multiple respective scanning cycles, and the imaging processor produces multiple respective super-resolution images corresponding to the to the scanning cycles, e.g., using the method described above. In an embodiment, the imaging processor estimates based on the multiple super-resolution images at least one hemodynamic parameter of the target, such as blood flow, blood velocity and blood volume.

In some embodiments, the imaging processor handles the IQ images by dividing them into overlapping sub-blocks, each sub-block comprising, for example, 64-by-64 pixels. In such embodiments, the sparsity-based resolver recovers the respective multiple super-resolution sub-blocks, and stitches the recovered super-resolution sub-blocks together to produce the super-resolved image.

### Efficient Sparse Recovery Using FFT Operations

As noted above, the model matrix A used for sparse recovery typically consumes a large storage space. In this section we present efficient implementation of sparsity-based resolver 64 that does not require explicit calculation and storage of A. Instead, small-sized data structures are pre-calculated, and used in multiplication operations of the form Ax using FFT operations. Other suitable ways to perform optimization efficiently are also possible and can be applied to transform domains other than the frequency domain using FFT.

Next we describe an iterative process for solving the optimization problem of Equation 24 above. The iterative process solves the convex optimization by updating a gradient value of the function ƒ(x)=½∥y−Ax∥₂² in each iteration.

The main signal-based input to the iterative process is y=y_{d}, as calculated, for example, at step 120 of the method of FIG. 4. For simplicity we omit the Doppler band indexing.

Input parameters for the iterative process comprise a regularization parameter λ≥0 and the maximal number of iterations K_{MAX}. The number of iterations can be set to several tens, e.g., 250 iterations.

The sparsity-based resolver initializes auxiliary variables as follows: z₁=x₀=0, t₁=1, and k=1. Then the sparsity-based resolver carries out processing iterations as described in the pseudo-code below:

$$While k < {K}_{MAX} do \text{:}$$

$$1. Calculate {g}_{k} = {A}^{T} {Az}_{k} - {A}^{T} y . \\ 2. {x}_{k} = max ( \vert {z}_{k} - \frac{1}{{L}_{f}} {g}_{k} \vert - \frac{λ}{{L}_{f}} , 0 ) \cdot sign ( {z}_{k} - \frac{1}{{L}_{f}} {g}_{k} )$$

$$3. {t}_{k + 1} = 0.5 ( 1 + \sqrt{1 + 4 {t}_{k}^{2}} )$$

$$4. {z}_{k + 1} = {x}_{k} + \frac{{t}_{k - 1}}{{t}_{k + 1}} ( {x}_{k} - {x}_{k - 1} )$$

$$5. k ← k + 1$$

When the iteration loop over steps 1-5 terminates, the sparsity-based resolver outputs the most recent vector xₖ as the super-resolution solution. In the pseudo-code above, the loop runs over a predefined number of iterations. Alternatively or additionally, any other loop termination criteria can also be used.

In the pseudo-code above, L_{ƒ} is the Lipschitz constant, which equals the maximum eigenvalue of A^{T}A.

The most computationally intensive part of the iterative process is the calculation of the gradient value gₖ of ƒ(x) in 1. In some embodiments, the term A^{T}y is calculated once for a given input, and stored in memory, e.g., as part of initialization. A^{T}y is a vector of length M², which is much smaller than the size of A—M²-by-N².

The term A^{T}A has a size of N²-by-N², which is typically too large to be stored in memory and/or to be used for multiplication with an N²-by-1 vector. It can be shown that A^{T}A has a special Block Circulant with Circulant Blocks (BCCB) structure. Based on the BCCB structure, the sparsity-based resolver stores in memory a vector of N² eigenvalues of A^{T}A, and calculates A^{T}Azₖ efficiently using FFT and inverse FFT operations.

Detailed description of the described iterative method and other variants are described, for example, by Solomon at al. in “SPARCOM: Sparsity Based Super-Resolution Correlation Microscopy,” arXiv Preprint, volume 1707, number 9255, 2017, pages 1-14, which is incorporated herein by reference.

The embodiments described above are given by way of example, and other suitable embodiments can also be used. For example, although the embodiments described above refer mainly to processing ultrasound echoes reflected from UCAs such as microbubbles, the disclosed techniques are similarly applicable to imaging based echoes reflected from red blood cells (without injecting any contrast agents) or based on echoes reflected from both red blood cells and contrast agents. The disclosed techniques are applicable in imaging blood vessels in a variety of organs. In the embodiments described above we mainly refer to 2D images comprising 2D pixels. The disclosed embodiments are applicable, however, also to 3D images in which 3D voxels replace the 2D pixels.

The embodiments described above refer mainly to sparse-recovery processing in the frequency domain, by transforming the aggregated image using 2d-DFT. Alternatively, the sparse-recovery could also be formulated in the spatial domain using the aggregated image itself or in other domains. Alternative sparse recovery methods can also be used.

The embodiments described above refer mainly to applying a sparse-recovery function to an image aggregated over multiple input images. In alternative embodiments, the sparse-recovery function is applied to a series of input images to produce respective temporary super-resolution images, which are aggregated to produce the output super-resolution image.

Although the embodiments described herein mainly address ultrasound imaging, the methods and systems described herein can also be used in other applications, such as in Photoacoustic imaging that combines optical excitation and ultrasound imaging of blood cells. The disclosed techniques are also applicable in imaging of static contrast agents such as nano-droplets that are activated using ultrasound, and the signal they produce changes with time even though the locations of the nano-droplets are fixed.

It will be appreciated that the embodiments described above are cited by way of example, and that the following claims are not limited to what has been particularly shown and described hereinabove. Rather, the scope includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An apparatus for imaging, comprising:
an input interface, configured to receive a sequence of input images of a target, wherein each input image comprises a grid of pixels representing reflections of a transmitted signal from reflectors or scatterers in the target, and wherein a resolution of the input images is degraded by a measurement process of capturing the input images in the sequence; and
a processor, configured to:
derive, from the sequence of input images, an aggregated image in which each pixel comprises a statistical moment calculated over corresponding pixels of the input images; and
convert the aggregated image into a super-resolution image of the target, having a higher resolution than the input images, by applying to the aggregated image a recovery function, which outputs the super-resolution image as a solution to the recovery function, provided that the reflectors or scatterers are sparse or compressible in the target in a predefined transform domain.

2. The apparatus according to claim 1, wherein the target comprises a vasculature of an organ, and wherein the reflectors or scatterers comprise one or more of (i) microbubbles administered into the vasculature and (ii) red blood cells flowing within the vasculature.

3. The apparatus according claim 2, wherein the processor is configured to convert the aggregated image into the super-resolution image so that reflectors or scatterers corresponding to overlapping echoes appear visually separated in the super-resolution image.

4. The apparatus according to claim 2, wherein the processor is configured to:
derive from the sequence of input images multiple Doppler-band-specific image sequences, based on identifying multiple respective ranges of microbubble velocities;
aggregate each of the Doppler-band-specific image sequences to produce a Doppler-band-specific aggregated image;
convert each of the Doppler-band-specific aggregated images into a respective Doppler-band-specific super-resolution image; and
reconstruct the super-resolution image of the target from the multiple Doppler-band-specific super-resolution images.

5. The apparatus according to claim 1, wherein the processor is configured to transform the aggregated image from a spatial domain to a transform domain using a predefined transform, and to apply the recovery function to the aggregated image in the transform domain.

6. The apparatus according to claim 1, wherein the aggregated image and the super-resolution image are interrelated using a model that depends on a Point Spread Function (PSF) included in the measurement process.

7. The apparatus according to claim 6, wherein the processor is configured to estimate the PSF by identifying in the input images regions corresponding to non-overlapping echoes of the reflectors or scatterers, and estimating the PSF based on the identified regions.

8. The apparatus according to claim 6, wherein the model comprises an underdetermined linear model, wherein the predefined recovery function comprises a convex optimization problem based on the linear model, and wherein the processor is configured to solve the convex optimization problem under a sparsity constraint.

9. The apparatus according to claim 8, wherein a matrix formulating the linear model has a Block Circulant with Circulant Blocks (BCCB) structure, and wherein the processor is configured to solve the convex optimization problem by performing a sequence of iterations, and based on the BCCB structure, to calculate in each iteration a gradient value of a function derived from the linear model using FFT-based operations.

10. The apparatus according to claim 8, wherein the optimization problem is formulated under a Total Variation (TV) constraint.

11. The apparatus according to claim 8, wherein the optimization problem is formulated in a selected domain in which the solution is sparse and wherein the processor is configured to solve the optimization problem in the selected domain.

12. The apparatus according to claim 1, wherein the input interface is configured to receive multiple sequences of the input images over multiple respective scanning cycles, and wherein the processor is configured to produce multiple respective super-resolution images corresponding to the to the scanning cycles, and to estimate based on the multiple super-resolution images at least one hemodynamic parameter of the target.

13. The apparatus according to claim 1, wherein the recovery function comprises an optimization problem selected from a list consisting of: a sparse-recovery function, a compressible-recovery function, and a regularized-recovery function.

14. A method for imaging, comprising:
receiving a sequence of input images of a target, wherein each input image comprises a grid of pixels representing reflections of a transmitted signal from reflectors or scatterers in the target, and wherein a resolution of the input images is degraded by a measurement process of capturing the input images in the sequence;
deriving, from the sequence of input images, an aggregated image in which each pixel comprises a statistical moment calculated over corresponding pixels of the input images; and
converting the aggregated image into a super-resolution image of the target, having a higher resolution than the input images, by applying to the aggregated image a recovery function, which outputs the super-resolution image as a solution to the recovery function, provided that the reflectors or scatterers are sparse or compressible in the target in a predefined transform domain.

15. The method according to claim 14, wherein the target comprises a vasculature of an organ, and wherein the reflectors or scatterers comprise one or more of (i) microbubbles administered into the vasculature and (ii) red blood cells flowing within the vasculature.

16. The method according claim 15, wherein converting the aggregated image comprises converting the aggregated image into the super-resolution image so that reflectors or scatterers corresponding to overlapping echoes appear visually separated in the super-resolution image.

17. The method according to claim 15, and comprising deriving from the sequence of input images multiple Doppler-band-specific image sequences, based on identifying multiple respective ranges of microbubble velocities, aggregating each of the Doppler-band-specific image sequences to produce a Doppler-band-specific aggregated image, converting each of the Doppler-band-specific aggregated images into a respective Doppler-band-specific super-resolution image, and reconstructing the super-resolution image of the target from the multiple Doppler-band-specific super-resolution images.

18. The method according to claim 14, wherein converting the aggregated image comprises transforming the aggregated image from a spatial domain to a transform domain using a predefined transform, and wherein applying the recovery function comprises applying the recovery function to the aggregated image in the transform domain.

19. The method according to claim 14, wherein the aggregated image and the super-resolution image are interrelated using a model that depends on a Point Spread Function (PSF) included in the measurement process.

20. The method according to claim 19, and comprising estimating the PSF by identifying in the input images regions corresponding to non-overlapping echoes of the reflectors or scatters, and estimating the PSF based on the identified regions.

21. The method according to claim 19, wherein the model comprises an underdetermined linear model, wherein the recovery function comprises a convex optimization problem based on the linear model, and wherein applying the recovery function comprises solving the convex optimization problem under a sparsity constraint.

22. The method according to claim 21, wherein a matrix formulating the linear model has a Block Circulant with Circulant Blocks (BCCB) structure, and wherein solving the convex optimization problem comprises performing a sequence of iterations, and based on the BCCB structure, calculating in each iteration a gradient value of a function derived from the linear model using FFT-based operations.

23. The method according to claim 21, wherein the optimization problem is formulated under a Total Variation (TV) constraint.

24. The method according to claim 21, wherein the optimization problem is formulated in a selected domain in which the solution is sparse, and wherein solving the optimization problem comprises solving the optimization problem in the selected domain.

25. The method according to claim 14, and comprising receiving multiple sequences of the input images over multiple respective scanning cycles, producing multiple respective super-resolution images corresponding to the to the scanning cycles, and estimating based on the multiple super-resolution images at least one hemodynamic parameter of the target.

26. The method according to claim 14, wherein the recovery function comprises an optimization problem selected from a list consisting of: a sparse-recovery function, a compressible-recovery function, and a regularized-recovery function.

27. An apparatus for imaging, comprising:
an input interface, configured to receive a series of input images of a target, wherein each input image comprises a grid of pixels representing reflections of a transmitted signal from reflectors or scatterers in the target, and wherein a resolution of the input images is degraded by a measurement process of capturing the input images in the series; and
a processor, configured to:
convert the input images in the series into respective temporary super-resolution images of the target, having a higher resolution than the input images, by applying to each of the input images a recovery function, which outputs the respective temporary super-resolution image as a solution of the recovery function, provided that the reflectors or scatterers are sparse or compressible in the target in a predefined transform domain; and
reconstruct an output super-resolution image of the target by aggregating the temporary super-resolution images.

28. A method for imaging, comprising:
receiving a series of input images of a target, wherein each input image comprises a grid of pixels representing reflections of a transmitted signal from reflectors or scatterers in the target, and wherein a resolution of the input images is degraded by a measurement process of capturing the input images in the series;
converting the input images in the series into respective temporary super-resolution images of the target, having a higher resolution than the input images, by applying to each of the input images a recovery function, which outputs the respective temporary super-resolution image as a solution to the recovery function, provided that the reflectors or scatterers are sparse or compressible in the target in a predefined transform domain; and
reconstructing an output super-resolution image of the target by aggregating the temporary super-resolution images.
